(19) United States
(12) Patent Application Publication
(10) Pub. No.: US 2021/0345960 A1
(43) Pub. Date: Nov. 11, 2021

(54) BODY WEIGHT ESTIMATION DEVICE, BODY WEIGHT ESTIMATION METHOD, AND PROGRAM RECORDING MEDIUM

(71) Applicant: NEC Corporation, Minato-ku, Tokyo (JP)

(72) Inventors: Chenhui HUANG, Tokyo (JP); Kenichiro FUKUSHI, Tokyo (JP); Hiroshi KAJITANI, Tokyo (JP); Kentaro NAKAHARA, Tokyo (JP); Takeo NOZAKI, Tokyo (JP)

(73) Assignee: NEC Corporation, Minato-ku, Tokyo (JP)

(21) Appl. No.: 17/283,989

(22) PCT Filed: Oct. 17, 2018
(86) PCT No.: PCT/JP2018/038695

§ 371 Date: Apr. 9, 2021

(51) Int. Cl. A61B 5/00; A61B 5/11; A61B 5/103

(52) U.S. Cl. CPC A61B 5/4869; A61B 5/112; A61B 2562/0247; A61B 5/7246; A61B 5/1038; A61B 5/7267

(57) ABSTRACT

A body weight estimation device includes: a data reception unit that receives gait data including the gait characteristics of a walking person; a first calculation unit that extracts a feature quantity based on the gait characteristics of the walking person from the gait data; a second calculation unit that generates a learning model by learning a correlation between the feature quantity extracted by the first calculation unit and body weight information about the walking person, using the gait data as sample data; and an estimation unit that estimates the body weight information associated with the gait data of an estimation target by inputting the gait data of the estimation target to the learning model.

### TECHNICAL FIELD

The present invention relates to a body weight estimation device, a body weight estimation method, and a program for estimating the body weight of a walking person.

### BACKGROUND ART

Growing interest in health has brought attention to the use of a digital health technology to monitor biomarkers, such as pulse waves, heart rate, blood pressure, body temperature, muscle activity, and body weight, which are physical quantities acquired from a living human body.

In this context, there is growing interest in monitoring weight change as a biomarker using a digital health technology.

In general body weight measurement, a gravitational force a human body in a resting state receives is measured with a weight scale that measures body weight in accordance with the principles of spring expansion/contraction and strain deformation. However, when body weight is measured with a weight scale, it is necessary for a person to stand on a weight scale placed on the ground, and therefore, the environments such as time and space in which body weight can be measured are limited. For example, in a case where body weight is monitored during health control, it is necessary to perform measurement at a fixed time and place, because information cannot be obtained unless a weight scale is installed in the environment. Therefore, conventional body weight measurement methods put burdens on those who need to monitor their weight at a high frequency, such as once a day or three times a day, and may lower motivation for health control. In view of this, there is an increasing demand for a technique for measuring body weight at any time in daily life.

PTL 1 discloses a sheet-like load measuring device that can be installed as an insole of a shoe. The device disclosed in PTL 1 includes: a sensor unit including two capacitors formed by sandwiching a sheet-like dielectric material with voids and dents and a flat sheet-like dielectric material between three sheet-like conductors; and an electric circuit unit operates to measure changes in the capacitance difference between the capacitors of the sensor unit.

PTL 2 discloses a path shape determining device that includes: a pattern acquisition unit that acquires a motion pattern of a walking user; and a path shape determination unit that determines the shape of the path on which the user has walked.

PTL 3 discloses a weight information output system that includes:

an acquisition unit that acquires acceleration information about a living subject; and an output unit that outputs weight information about the living subject, the weight information being a weight corresponding to a feature quantity related to an acceleration change in the living subject identified from the acceleration information.

PTL 4 discloses an individual identification device that identifies an individual based on displacement of an electric field formed in a human body during gait motion. The device disclosed in PTL 4 includes a field displacement detecting means for detecting displacement of an electric field formed in a human body while the human body is moving on two feet. The device disclosed in PTL 4 also includes an identifying means for identifying an individual by using an index that is a peak of an amplitude relating to a predetermined frequency band associated with a state in which the entire sole of one foot is in contact with the ground, and the other toe has just left the ground, in the displacement of the electric field.

PTL 5 discloses a balance sense measuring device that measures a balance sense of a person at a time of walking. The device disclosed in PTL 5 measures the balance sense of a walking person, from the deflection or twisting caused in a walking beam by a person walking on the walking beam stretched between a pair of supporting stands, and the time of passage.

### CITATION LIST

### Patent Literature

[PTL 1] JP 4860430 B1

[PTL 2] JP 6054905 B1

[PTL 3] JP 2017-211304 A

[PTL 4] JP 2004-147793 A

[PTL 5] JPH 06-245924 A

### SUMMARY OF INVENTION

### Technical Problem

The device disclosed in PTL 2 can calculate a body weight on the basis of a change in the pressure of the sole after a certain time measured by a pressure sensor attached to the inside of the shoe, and the initial body weight. However, the device disclosed in PTL 2 has a problem that measurement accuracy is low, because the device is incapable of determining whether a pressure change detected by the pressure sensor is due to a change in body weight, or whether the pressure change is due to movement of the body.

The device disclosed in PTL 3 focuses on changes in acceleration during walking, and estimates a body weight from the relationship between the acceleration and the changes in body weight. The acceleration generated during walking is caused by the forward and backward movement of the lower limbs. Since the lower limbs move around the pelvis, changes in acceleration during walking match the waveforms for recording movement of the lower limbs. In other words, the device disclosed in PTL 3 is capable of detecting changes in movement of the lower limbs during walking, but is not capable of measuring changes in body weight during walking, which is a problem. Further, when the position of installation of the device disclosed in PTL 3 changes, the motion characteristics of the lower limbs change, and the acceleration characteristics also change. As a result, a model relationship between acceleration characteristics and body weight cannot be used, and therefore, body weight estimation accuracy drops, which is a problem.

The device disclosed in PTL 4 focuses on changes in the capacitance of a human body during walking, and extracts feature quantities from gait waveforms. However, the device disclosed in PTL 4 has a problem that it is difficult to determine whether a change in the capacitance of a human body is caused by walking, or whether the change is caused by some other body movement.

The device disclosed in PTL 5 can measure the body weight of a walking person, on the basis of an output signal of a strain gauge installed on a walking beam. However, the device disclosed in PTL 5 can evaluate the balance sense of a walking person on the basis of deflection or twisting of the walking beam, but cannot measure the body weight of the walking person with high accuracy, which is a problem.

The present invention aims to solve the above problems, and provide a body weight estimation system capable of estimating the body weight of a walking person with high accuracy.

### Solution to Problem

A body weight estimation device of one aspect of the present invention includes: a data reception unit that receives gait data including the gait characteristics of a walking person; a first calculation unit that extracts a feature quantity based on the gait characteristics of the walking person from the gait data; a second calculation unit that generates a learning model by learning a correlation between the feature quantity extracted by the first calculation unit and body weight information about the walking person, using the gait data as sample data; and an estimation unit that estimates the body weight information associated with the gait data of an estimation target by inputting the gait data of the estimation target to the learning model.

A body weight estimation method of one aspect of the present invention includes: receiving gait data including gait characteristics of a walking person; extracting a feature quantity based on the gait characteristics of the walking person from the gait data; generating a learning model by learning a correlation between the extracted feature quantity and body weight information about the walking person, using the gait data as sample data; and estimating the body weight information associated with the gait data of an estimation target by inputting the gait data of the estimation target to the learning model.

A program of one aspect of the present invention is a program for causing a computer to perform: a process of receiving gait data including the gait characteristics of a walking person; a process of extracting a feature quantity based on the gait characteristics of the walking person from the gait data; a process of generating a learning model by learning a correlation between the feature quantity extracted by the first calculation unit and body weight information about the walking person, using the gait data as sample data; and a process of estimating the body weight information associated with the gait data of an estimation target, by inputting the gait data of the estimation target to the learning model.

### Advantageous Effects of Invention

According to the present invention, it is possible to provide a body weight estimation system capable of estimating the body weight of a walking person with high accuracy.

### BRIEF DESCRIPTION OF DRAWINGS

FIG. 1 is a block diagram showing an example configuration of a body weight estimation system according to a first example embodiment of the present invention.

FIG. 2 is a conceptual diagram for explaining a gait cycle of a person.

FIG. 3 is a graph showing an example of temporal changes in foot pressure measured when a person is walking.

FIG. 4 is a conceptual diagram showing an example of a load measurement device of the body weight estimation system according to the first example embodiment of the present invention.

FIG. 5 is a conceptual diagram showing an example of the data acquisition device of the body weight estimation system according to the first example embodiment of the present invention.

FIG. 6 is a conceptual diagram showing an example of a body weight estimation device of the body weight estimation system according to the first example embodiment of the present invention.

FIG. 7 is a flowchart for explaining an example operation of the data collection device of the body weight estimation system according to the first example embodiment of the present invention.

FIG. 8 is a flowchart for explaining an example operation of a first calculation unit of the body weight estimation device of the body weight estimation system according to the first example embodiment of the present invention.

FIG. 9 is an example of feature quantity vectors extracted by the first calculation unit of the body weight estimation device of the body weight estimation system according to the first example embodiment of the present invention.

FIG. 10 is a flowchart for explaining an example operation of a second calculation unit of the body weight estimation device of the body weight estimation system according to the first example embodiment of the present invention.

FIG. 11 is a flowchart for explaining an example operation of a body weight estimation unit of the body weight estimation device of the body weight estimation system according to the first example embodiment of the present invention.

FIG. 12 is a block diagram showing an example configuration of a body weight estimation system according to a second example embodiment of the present invention.

FIG. 13 is a flowchart showing an example of procedures for recording feature quantities in an example of the present invention.

FIG. 14 is an example of gait waveforms recorded in an example of the present invention.

FIG. 15 is a graph showing the correlation between true body weights and predicted body weights obtained in an example of the present invention.

FIG. 16 is a block diagram showing an example hardware configuration for obtaining a body weight estimation system according to each example embodiment of the present invention.

### EXAMPLE EMBODIMENTS

The following is a description of example embodiments of the present invention, with reference to the drawings. Although the example embodiments described below are technically preferable for carrying out the present invention, the scope of the invention is not limited to them. In all the drawings to be used in conjunction with the description of the following example embodiments, like components are denoted by like reference numerals, unless there is a particular reason. Further, in the example embodiments described below, explanation of like components and operations may not be repeated.

### First Example Embodiment

First, a body weight estimation system according to a first example embodiment of the present invention is described, with reference to drawings. The body weight estimation system of this present example embodiment estimates the body weight of a user, using gait data of the user. In general, gait refers to the manners in which humans and animals walk. The present example embodiment relates to the gait of a person. Gait includes a stride length (right or left, equivalent to one step), a stride length (two steps), rhythm, speed, dynamic basis, traveling direction, foot angle, hip angle, and ability to crouch down. The body weight estimation device of the present example embodiment acquires gait data by measuring temporal changes in the loads (also called the load waveforms) of the soles of the user, extracts the feature quantities of the gait data, and estimates the body weight of the user. In the description below, the term “walking person” refers mainly to a user who is walking, but a user who has stopped may also be referred to as a walking person.

### Configuration

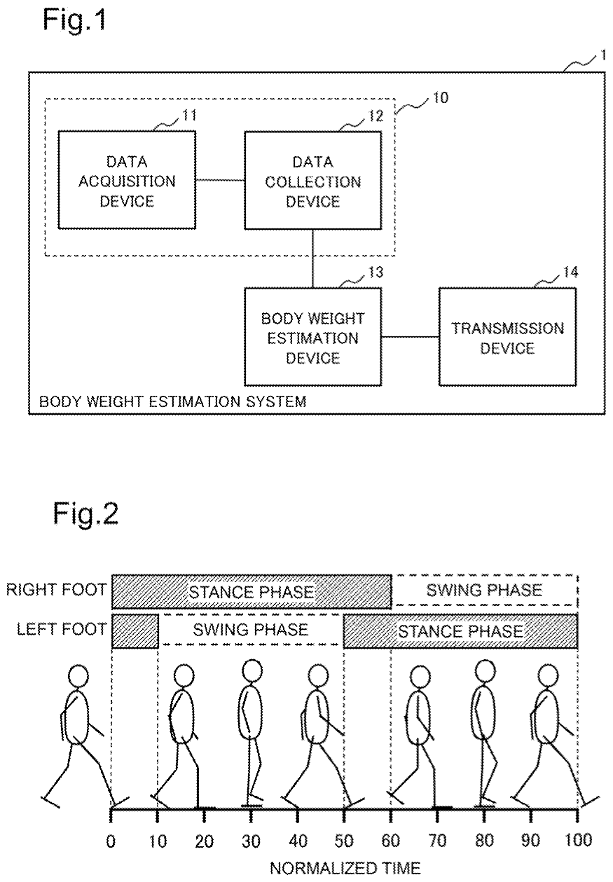

FIG. 1 is a block diagram showing a schematic configuration of a body weight estimation system 1 according to the present example embodiment. As shown in FIG. 1, the body weight estimation system 1 includes a data acquisition device 11, a data collection device 12, a body weight estimation device 13, and a transmission device 14. The data acquisition device 11 and the data collection device 12 constitute a gait data generation unit 10.

The data acquisition device 11 is a device that measures loads received from the soles of the user. The data acquisition device 11 includes at least one pressure sensor that measures loads received from the soles of the user. The data acquisition device 11 transmits sensor data detected by the pressure sensor(s) to the data collection device 12. The data acquisition device 11 may individually transmit the sensor data that is measured by each pressure sensor and is associated with the measurement point, or collectively transmit all the sensor data.

The data collection device 12 receives the sensor data detected by the data acquisition device 11. The data collection device 12 extracts gait data from the received sensor data. The data collection device 12 stores the extracted gait data. In a learning mode, body weight information about the user is input to the data collection device 12 in conjunction with the input of the sensor data. The body weight information is information including the body weight of the user. In a case where the body weight information about the user is input to the data collection device 12, the data collection device 12 stores the input body weight information associated with the gait data acquired in conjunction with the body weight information.

The body weight estimation device 13 is connected to the data collection device 12 by wired or wireless communication. The body weight estimation device 13 receives the gait data from the data collection device 12. The body weight estimation device 13 determines the state of the user, on the basis of the received gait data. When having determined that the user is walking on the basis of the gait data, the body weight estimation device 13 extracts a feature quantity from the gait data. In the learning mode, the body weight estimation device 13 stores the extracted feature quantity. The body weight estimation device 13 generates a learning model by learning the correlation between the characteristics indicated by the gait data and body weight information, and stores the generated learning model. In a measurement mode, the body weight estimation device 13 estimates body weight data, using the feature quantity extracted from the gait data of the estimation target and the learning model. The body weight estimation device 13 transmits the estimated body weight data to the transmission device 14.

The transmission device 14 is connected to the body weight estimation device 13. The transmission device 14 acquires the data to be transmitted to the outside from the body weight estimation device 13. The transmission device 14 transmits the data to a device having a display unit, such as a stationary information processing device like a personal computer or a server, a mobile terminal like a smartphone or a mobile phone, or a display device. The transmission mode of the transmission device 14 may be wired communication via a cable or wireless communication using electromagnetic waves, acoustic waves, or the like, and the transmission mode is not limited to any particular one. For example, the user can detect a situation of and a change in his/her body weight by referring to the body weight data displayed on a monitor.

The outline of the configuration of the body weight estimation system 1 has been described so far. However, the configuration of the body weight estimation system 1 shown in FIG. 1 is merely an example, and the configuration of the body weight estimation system 1 according to the present example embodiment does not need to remain as it is.

[Gait Cycle]

A human gait cycle is now described, with reference to a drawing. FIG. 2 is a conceptual diagram for explaining a gait cycle of a person, with the right foot being the reference. The abscissa axis shown under the walking person in FIG. 2 indicates the normalized time obtained by normalizing the elapsed time accompanying the gait of the person. In the description below, explanation focuses on the right foot, but the same explanation applies to the left foot.

A gait cycle of a person is roughly divided into a stance phase and a swing phase. The stance phase of the right foot is the period that starts when the heel of the right foot comes into contact with the ground, lasts while the sole of the left foot is completely in contact with the ground, and ends when the toe of the right foot leaves the ground. The stance phase accounts for 60% of the total gait cycle. The swing phase of the right foot is the period that starts when the sole of the left foot is completely in contact with the ground and the toe of the right foot leaves the ground, and lasts until the heel of the right foot again comes into contact with the ground. The swing phase accounts for 40% of the total gait cycle.

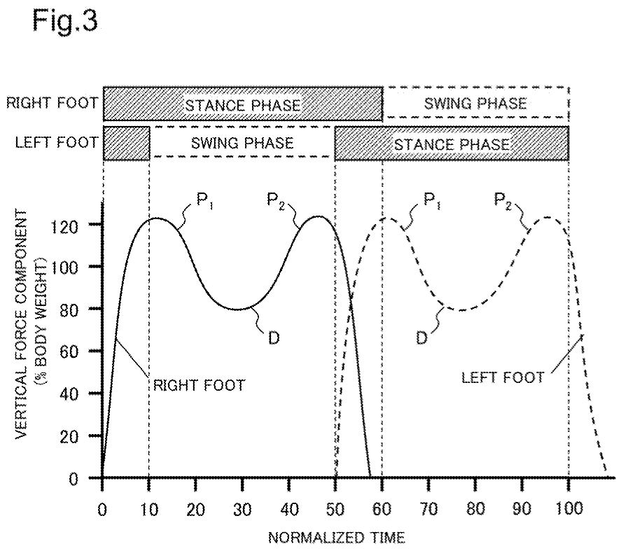

FIG. 3 is a graph showing an example of temporal changes in foot pressure (pressure on the sole of the foot) measured when a person is walking. The abscissa axis in FIG. 3 indicates the normalized time obtained by normalizing the passage of time accompanying the gait of the person, and corresponds to the abscissa axis in FIG. 2. As for the curves shown in FIG. 3, the solid line indicates the time transition of the foot pressure of the right foot, and the dashed line indicates the time transition of the foot pressure of the left foot.

The time transition (the solid line) of the vertical component of force of the right foot during walking shows two peaks (a first peak P₁ and a second peak P₂) and a valley (a dip D). For example, the first peak P₁, the second peak P₂, and the dip D can be separated into waveforms representing these peaks and the dip. The first peak P₁ is caused by the impact generated when the entire sole of the foot comes into contact with the ground due to the vertical rotation of the ankle joint after the heel of the right foot touches the ground. The second peak P₂ is caused by the pressure exerted on the ground by the toe of the right foot when the person's posture leans forward as the heel of the left foot touches the ground and the toe of the right foot leaves the ground between the end of the stance phase and the start of the swing phase of the right foot. The value of the foot pressure at the apex of the second peak P₂ corresponds to the value obtained by adding the load applied by the body weight to the vertical component of force generated by the muscle when the walking person moves forward. The dip D is caused by the acceleration in the direction opposite from the direction of gravitational force caused by the upward movement of the left foot, which occurs in the middle of the stance phase of the right foot.

The first peak P₁, the second peak P₂, and the dip D included in the gait data are all related to the body weight of the user. Therefore, a learning model showing the correlation between the feature quantity extracted from the gait data (the first peak P₁, the second peak P₂, and the dip D, for example) and the body weight is generated in advance. Thus, it is possible to estimate the body weight by inputting the gait data of the estimation target to the learning model.

A gait cycle of a person has been described so far. However, the gait cycle of a person shown in FIGS. 2 and 3 is merely an example, and does not limit gait cycles to be examined by the body weight estimation system 1 of the present example embodiment.

Next, the configurations of the data acquisition device 11, the data collection device 12, and the body weight estimation device 13 are described, with reference to drawings.

[Data Acquisition Device]

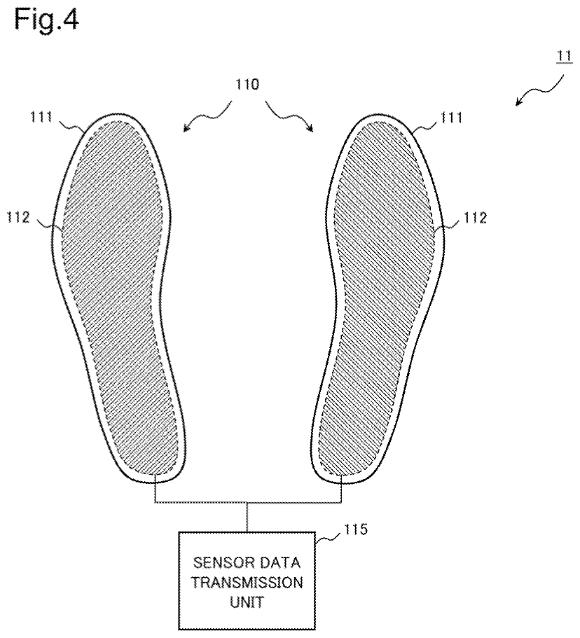

FIG. 4 is a conceptual diagram showing an example configuration of the data acquisition device 11. As shown in FIG. 4, the data acquisition device 11 includes two data acquisition units 110 and a sensor data transmission unit 115. The data acquisition units 110 each includes a main frame 111 and a sensor unit 112. The data acquisition units 110 are installed as insoles in shoes and are used.

Each main frame 111 has the external shape of a shoe insole. The main frames 111 may have different shapes for the left foot and the right foot, or may have the same shape. Further, the main frames 111 may be made of a general material for insoles, or may be made of a material with enhanced rigidity and functionality. For example, each main frame 111 has a stacked structure formed with at least two layers, and the sensor unit 112 is inserted between two of the layers.

The sensor units 112 are installed in or on surfaces of the main frames 111. The sensor units 112 are connected to the sensor data transmission unit 115. The sensor units 112 each includes at least one sensor, and detects a physical quantity related to the walking manner of the person, and changes in the physical quantity. The sensor units 112 output sensor data based on the detected physical quantities and changes therein, to the sensor data transmission unit 115.

For example, each sensor unit 112 detects physical quantities related to foot pressure, foot pressure distribution, acceleration, angular velocity, myoelectric strength, and the like, and changes in the physical quantities. For example, each sensor unit 112 can be formed with a pressure sensor that detects a pressure received from a sole of the user wearing shoes for which the data acquisition device 11 is installed. Also, each sensor unit 112 can be formed with a sheet-like sensor sheet capable of measuring a pressure distribution. Using pressure sensor sheets as the sensor units 112, it is possible to measure distributions of pressure received from the soles. Each sensor unit 112 may be formed with a single sensor or a combination of a plurality of sensors. In a case where each sensor unit 112 is formed with a plurality of sensors, the sensor unit 112 may be formed with a plurality of sensors of the same type or a plurality of sensors of different types.

The sensor data transmission unit 115 is connected to the sensor units 112. The sensor data transmission unit 115 is also connected to the data collection device 12 by wired communication or wireless communication. For example, the sensor data transmission unit 115 is connected to the data collection device 12 by a wireless local area network (LAN), near field communication, or the like. The sensor data transmission unit 115 acquires sensor data from each sensor unit 112. The sensor data transmission unit 115 transmits the acquired sensor data to the data collection device 12. Although only one sensor data transmission unit 115 is shown in FIG. 4, sensor data transmission units 115 may be provided for the respective data acquisition units 110 for the left foot and the right foot.

The configuration of the data acquisition device 11 has been described so far. However, the configuration of the data acquisition device 11 shown in FIG. 4 is merely an example, and does not limit the configuration of the data acquisition device 11 according to the present example embodiment.

[Data Collection Device]

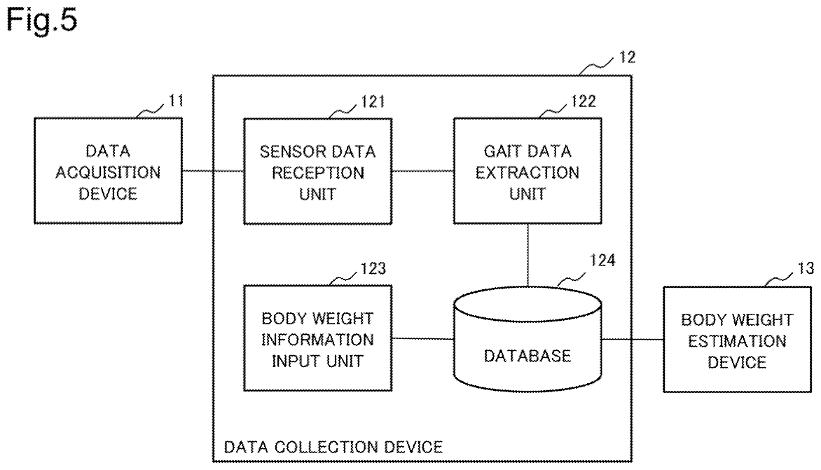

FIG. 5 is a block diagram showing an example configuration of the data collection device 12. As shown in FIG. 5, the data collection device 12 includes a sensor data reception unit 121, a gait data extraction unit 122, a body weight information input unit 123, and a database 124.

The sensor data reception unit 121 is connected to the sensor data transmission unit 115 of the data acquisition device 11 by wired or wireless communication. The sensor data reception unit 121 receives sensor data transmitted from the data acquisition device 11. The sensor data reception unit 121 outputs the received sensor data to the gait data extraction unit 122.

The gait data extraction unit 122 is connected to the sensor data reception unit 121. The gait data extraction unit 122 acquires the sensor data from the sensor data reception unit 121. The gait data extraction unit 122 extracts gait data from the acquired sensor data. For example, the gait data extraction unit 122 extracts data related to the manner of walking of the person, such as foot pressure, foot pressure distribution, acceleration, angular velocity, and myoelectric strength, as the gait data. The gait data extraction unit 122 stores the extracted gait data into the database 124.

The body weight information input unit 123 receives an input of body weight information from the user. For example, body weight information is input to the body weight information input unit 123 via a keyboard or a touch panel (not shown). Once body weight information is input, the body weight information input unit 123 associates the body weight information with the corresponding gait data, and stores the body weight information into the database 124.

The database 124 is connected to the gait data extraction unit 122. The database 124 is also connected to the body weight estimation device 13 by wired or wireless communication. The database 124 stores data related to walking in a specific region. A specific region is one of the biological regions of the user to be measured. In the present example embodiment, a sole is defined as a specific region. As for the data related to walking in the specific region, foot pressure data indicating the pressures applied by the sole to the sensor unit 112 at the times of foot separation and landing is stored as gait data in the database 124.

The configuration of the data collection device 12 has been described so far. However, the configuration of the data collection device 12 shown in FIG. 5 is merely an example, and does not limit the configuration of the data collection device 12 according to the present example embodiment.

[Body Weight Estimation Device]

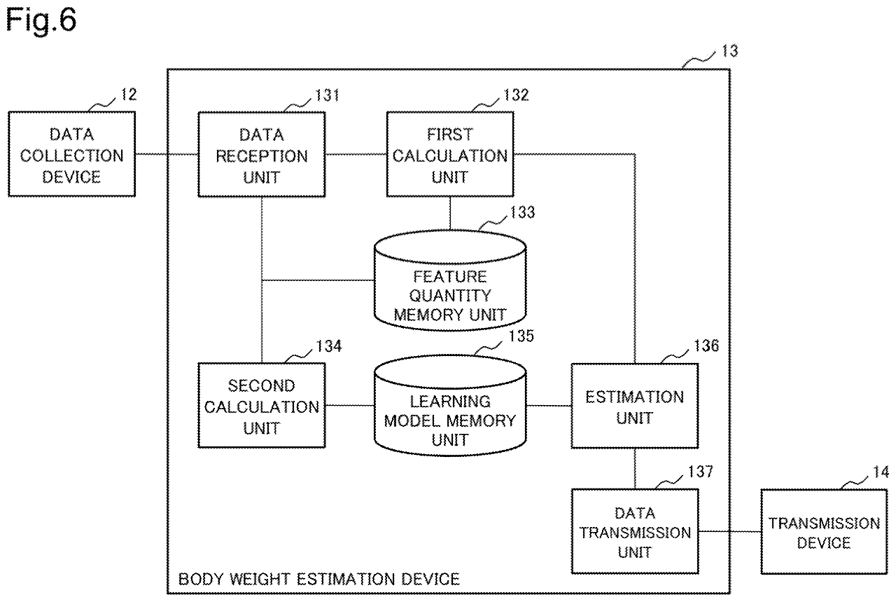

FIG. 6 is a block diagram showing an example configuration of the body weight estimation device 13. As shown in FIG. 6, the body weight estimation device 13 includes a data reception unit 131, a first calculation unit 132, a feature quantity memory unit 133, a second calculation unit 134, a learning model memory unit 135, an estimation unit 136, and a data transmission unit 137.

The data reception unit 131 acquires gait data acquired in a specific region from the database 124. For example, the data reception unit 131 acquires gait data related to the walking manner of a person, such as foot pressure that is primary data, foot pressure distribution, acceleration, angular velocity, and myoelectric strength. The data reception unit 131 outputs the acquired gait data to the first calculation unit 132.

The first calculation unit 132 acquires the gait data from the data reception unit 131. The first calculation unit 132 extracts a feature quantity indicating the characteristics of the gait from the acquired gait data. The first calculation unit 132 stores the extracted feature quantity into the feature quantity memory unit 133. In a case where body weight information is associated with the gait data, the feature quantity of the gait data is associated with the body weight information, and is then stored into the feature quantity memory unit 133. In a case where the first calculation unit 132 acquires the gait data of the estimation target, the first calculation unit 132 outputs the gait data of the estimation target to the estimation unit 136.

For example, the first calculation unit 132 extracts feature quantities such as walking speed, stride length, walking locus, balance between both feet, foot contact time, airborne time, stance phase time, and swing phase time, from the gait data that is primary data.

The first calculation unit 132 also extracts feature quantities from the time transition of the foot pressure during walking as shown in FIG. 3, for example. In the temporal change in the vertical component of force of the right foot during walking in FIG. 3, the first peak P₁ usually appears between 0% and 20% of the gait cycle, the dip D appears between 20% and 40% of the gait cycle, and the second peak P₂ appears between 40% and 60% of the gait cycle. For example, the feature quantities that can be extracted from the waveforms of temporal changes in the vertical component of force of the right foot during walking includes the values of the foot pressure at the apexes of the first peak P₁, the second peak P₂, and the dip D. The half widths of the first peak P₁, the second peak P₂, and the dip D, the sum, the average, the difference, the ratio, the product, and the time difference between at least two of these feature quantities, the integral value of the gait waveforms in the stance phase, and the like can also be extracted as feature quantities. As for the left foot, feature quantities can be extracted in the same manner as that for the right foot.

The feature quantity memory unit 133 (also called the first storage unit) stores the feature quantities relating to the gait extracted by the first calculation unit 132. The feature quantities stored in the feature quantity memory unit 133 are to be used by the second calculation unit 134 and the estimation unit 136. In a case where body weight information is associated with the gait data, the body weight information is associated with the feature quantities extracted from the gait data and is stored into the feature quantity memory unit 133.

The second calculation unit 134 acquires at least one piece of gait data as sample data from the data reception unit 131. The second calculation unit 134 generates a learning model by learning the correlation between the characteristics indicated by the acquired sample data and the body weight information. The second calculation unit 134 stores the generated learning model into the learning model memory unit 135.

For example, the second calculation unit 134 receives a plurality of pieces of sample data from the data reception unit 131, and performs machine learning to learn each piece of the sample data, using the feature quantities stored in the feature quantity memory unit 133 as teaching data. For example, the second calculation unit 134 uses a method such as a decision tree, a support vector machine, a neural network, a logistic regression, a nearest neighbor classification method, an ensemble classification learning method, and a discriminant analysis, to perform machine learning to learn each piece of the sample data.

For example, the second calculation unit 134 supplies each piece of the sample data to a support vector machine, and learns the relationship between the gait characteristics indicated by the first calculation unit 132 and the body weight data, such as the relationship between the body weight and the values of the foot pressure at the apexes of the first peak P₁, the second peak P₂, and the dip D. When the values of the foot pressure at the first peak P₁, the second peak P₂ and the dip D are input, the second calculation unit 134 generates a learning model for outputting the body weight data in accordance with the input values. The second calculation unit 134 stores the generated learning model into the learning model memory unit 135.

The second calculation unit 134 also performs deep learning using each piece of the sample data, and creates a classifier for determining the body weight data in accordance with the values of foot pressure at the apexes of the first peak P₁, the second peak P₂, and the dip D. The second calculation unit 134 stores the created classifier as a learning model into the learning model memory unit 135.

The learning model memory unit 135 (also called the secondary storage unit) stores the learning model generated by the second calculation unit 134. The learning model stored in the learning model memory unit 135 is to be used by the estimation unit 136.

The estimation unit 136 acquires the feature quantities of the gait data of the estimation target from the first calculation unit 132. Using the learning model stored in the learning model memory unit 135, the estimation unit 136 estimates the body weight of the user who is the acquirer of the gait data of the estimation target. The estimation unit 136 outputs body weight data indicating the estimated body weight to the data transmission unit 137.

The data transmission unit 137 acquires the body weight data from the estimation unit 136. The data transmission unit 137 transmits the acquired body weight data to the transmission device 14. The data transmission unit 137 may be designed to output the feature data stored in the feature quantity memory unit 133 to the transmission device 14.

The configuration of the body weight estimation device 13 according to the present example embodiment has been described so far. However, the configuration of the body weight estimation device 13 shown in FIG. 6 is merely an example, and does not limit the configuration of the body weight estimation device 13 according to the present example embodiment.

### Operations

Next, operations of the body weight estimation system 1 according to the present example embodiment are described. In the description below, an example of operations of the data collection device 12, and the first calculation unit 132, the second calculation unit 134, and the estimation unit 136 included in the body weight estimation device 13 are explained.

[Data Collection Device]

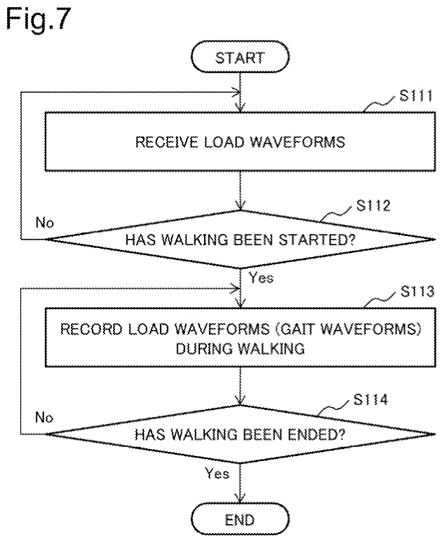

FIG. 7 is a flowchart for explaining an example operation of the data collection device 12. In the example described below with reference to the flowchart in FIG. 7, the data collection device 12 is the principal operator in the operation, and receives load waveforms as gait data from the data acquisition device 11. The load waveforms are the waveforms indicating temporal changes in the foot pressure acquired by the data acquisition device 11 when the user is walking.

In FIG. 7, the data collection device 12 receives load waveforms as gait data from the data acquisition device 11 (step S111).

The data collection device 12 then determines whether the user is walking, on the basis of the received load waveforms (step S112). For example, the data collection device 12 calculates the sum of all sensors included in the sensor units 112 of the data acquisition device 11, and determines whether to start walking from the temporal changes in the pressure value. It is also possible to set a threshold for detecting a sudden increase in the floor reaction force at the beginning of a stance phase, and determine that the point of time at which the floor reaction force exceeds the threshold is the start of walking, for example.

If it is determined that the user is walking (Yes in step S112), the data collection device 12 starts recording the load waveforms while the user is walking (step S113). The load waveforms recorded in step S113 are also referred to as gait waveforms. If the data collection device 12 determines that the user is not walking (No in step S112), on the other hand, the operation returns to step S111.

After step S113, the data collection device 12 determines whether the user has finished walking, on the basis of the received load waveforms (step S114). For example, the data collection device 12 determines that the end of the walking is the point of time at which a predetermined time has elapsed since the start of the walking or the point of time at which the gait waveforms reach a stable state for a certain time.

If the data collection device 12 determines that the user has finished walking (Yes in step S114), the process according to the flowchart in FIG. 7 comes to an end. If the data collection device 12 determines that the user has not finished walking (No in step S114), on the other hand, the operation returns to step S113, and the recording of the load waveforms is continued.

An example operation of the data collection device 12 has been described so far. However, the operation of the data collection device 12 according to the flowchart in FIG. 7 is merely an example, and does not limit operations of the data collection device 12 of the present example embodiment.

[First Calculation Unit]

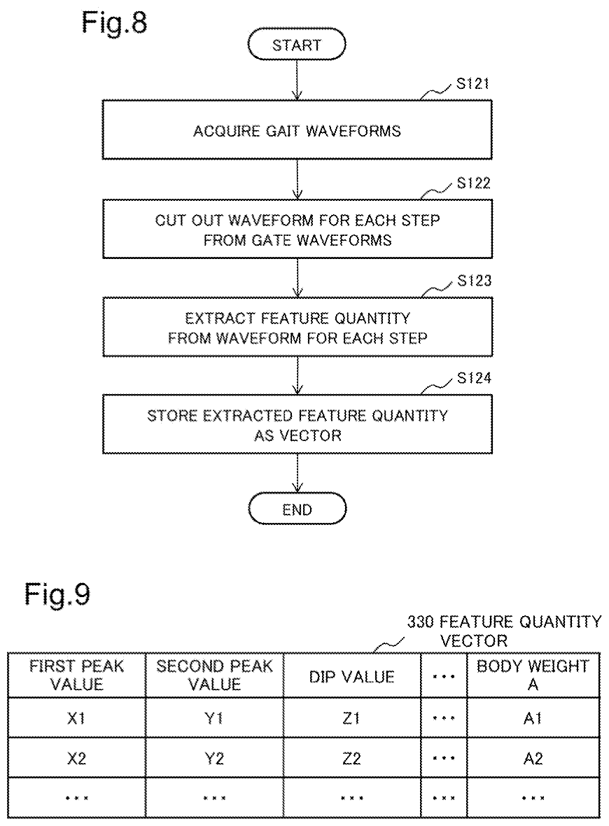

FIG. 8 is a flowchart for explaining an example operation of the first calculation unit 132 of the body weight estimation device 13. In the description below with reference to the flowchart in FIG. 8, the first calculation unit 132 is the principal operation in the operation.

In FIG. 8, the first calculation unit 132 first acquires gait waveforms from the data reception unit 131 (step S121).

The first calculation unit 132 then cut out the waveform of each step from the acquired gait waveforms (step S122). For example, the gait waveform shown in FIG. 3 rises rapidly after the start of a stance phase and falls rapidly before the start of a swing phase. Therefore, the range from the sudden rise to the sudden fall of the gait waveform can be identified as the waveform of one step.

The first calculation unit 132 then extracts feature quantities from the waveform of each step (step S123). To extract feature quantities from the waveform of each step (the right foot), the values of the foot pressure at the apexes of the first peak P₁, the second peak P₂, and the dip D appearing in the gait waveform in FIG. 3 are identified. Generally, the first peak P₁ appears between 0% and 20% of the gait cycle, the dip D appears between 20% and 40% of the gait cycle, and the second peak P₂ appears between 40% and 60% of the gait cycle. The maximum value of the foot pressure between 0% and 20% of the gait cycle, the minimum value of the foot pressure between 20% and 40% of the gait cycle, and the maximum value of the foot pressure between 40% and 60% of the gait cycle are identified, so that the values of the foot pressure at the apexes of the first peak P₁, the second peak P₂, and the dip D can be identified. Also, the values of the foot pressure at the apexes of the first peak P₁, the second peak P₂ and the dip D are identified, so that the times at which these apexes appear can be identified as occurrence times. The data between 60% and 100% of the gait cycle is regarded as the baseline. The values of the foot pressure at the apexes of the first peak P₁, the second peak P₂ and the dip D are combined and are subjected to the four basic arithmetic operations, so that feature quantities can be extracted.

The first calculation unit 132 then stores, into the feature quantity memory unit 133, the feature quantity vectors obtained by combining the feature quantities (explanatory variables) extracted by the component (the first calculation unit 132) and the body weight data (response variables) acquired by the second calculation unit 134 at the time of the creation of the learning model (step S124). FIG. 9 shows an example of feature quantity vectors 330 stored in the feature quantity memory unit 133 in step S124. The feature quantity vectors 330 include at least the following elements: the values of the foot pressure at the apexes of the first peak P₁, the second peak P₂ and the dip D, and body weights A.

An operation of the first calculation unit 132 has been described so far. However, the process described with reference to the flowchart in FIG. 8 is merely an example, and does not limit operations of the first calculation unit 132 of the present example embodiment.

[Second Calculation Unit]

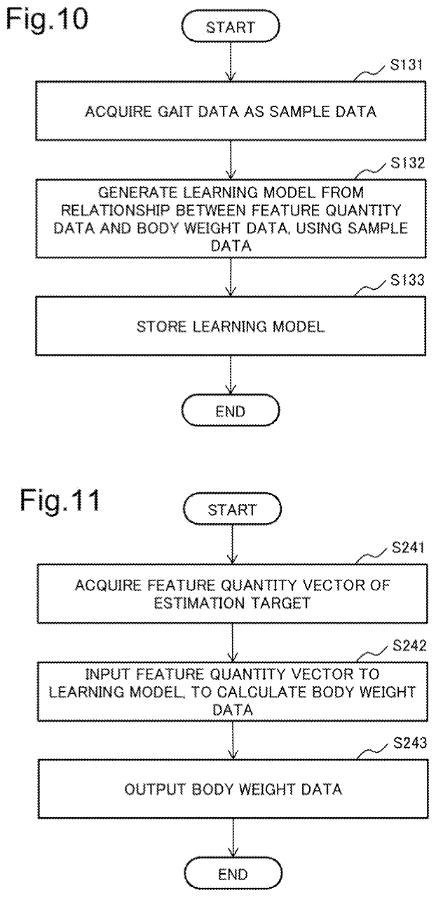

FIG. 10 is a flowchart for explaining an example operation of the second calculation unit 134 of the body weight estimation device 13. In the description below with reference to the flowchart in FIG. 10, the second calculation unit 134 is the principal operation in the operation.

In FIG. 10, the second calculation unit 134 first acquires gait data as sample data (step S131).

Using the sample data, the second calculation unit 134 generates a learning model from the relationship between the feature quantities extracted by the first calculation unit 132 and the body weight data (step S132).

The second calculation unit 134 then stores the generated learning model into the learning model memory unit 135 (step S133).

An operation of the second calculation unit 134 has been described so far. However, the process described with reference to the flowchart in FIG. 10 is merely an example, and does not limit operations of the second calculation unit 134 of the present example embodiment.

[Body Weight Estimation Unit]

FIG. 11 is a flowchart for explaining an example operation of the estimation unit 136 according to the first example embodiment of the present invention.

In FIG. 11, the estimation unit 136 first acquires the feature quantity vectors of the estimation target from the first calculation unit 132 (step S241).

The estimation unit 136 then inputs the acquired feature quantity vectors to the learning model stored in the learning model memory unit 135 (step S242).

The estimation unit 136 then outputs the calculated body weight data to the data transmission unit 137 (step S243). The body weight data output to the data transmission unit 137 is transmitted from the transmission device 14 to a user terminal. The feature quantity vectors may be transmitted together with the body weight data.

An operation of the estimation unit 136 has been described so far. However, the process described with reference to the flowchart in FIG. 11 is merely an example, and does not limit operations of the estimation unit 136 of the present example embodiment.

As described above, a body weight estimation system of the present example embodiment includes a data acquisition device, a data collection device, a body weight estimation device, and a transmission device. The body weight estimation system of the present example embodiment estimates the body weight of the user, taking into consideration of the influence of the body weight and the influence of body movement on the gait data related to temporal changes in the foot pressure whose measurement values hardly vary due to displacement of the installation position of the measuring instrument or the like. Accordingly, with the body weight estimation system of the present example embodiment, the body weight of a walking person can be estimated with high accuracy. In other words, with the body weight estimation system of the present example embodiment enables to estimate the body weight of the user without temporal and spatial restrictions, using the gait data acquired in specific regions.

### Second Example Embodiment

Next, a body weight estimation system according to a second example embodiment of the present invention is described, with reference to drawings. The body weight estimation system of the present example embodiment has the same configuration as that of the first example embodiment, except that the memory units and the transmission unit are removed from the body weight estimation device 13.

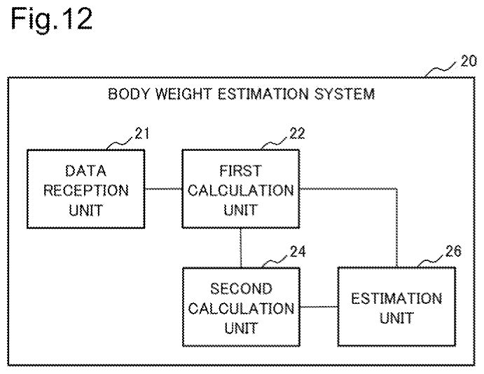

FIG. 12 is a block diagram showing an example configuration of a body weight estimation system 20 according to the present example embodiment. As shown in FIG. 12, the body weight estimation system 20 includes a data reception unit 21, a first calculation unit 22, a second calculation unit 24, and an estimation unit 26.

The data reception unit 21 receives gait data including the gait characteristics of a walking person. For example, the data reception unit 21 receives, as the gait data, data related to temporal changes in the pressure data of the soles of the walking person. For example, the data reception unit 21 receives, as the gait data, the load waveforms based on temporal changes in the pressure data the soles of the walking person.

The first calculation unit 22 extracts feature quantities based on the gait characteristics of the walking person from the gait data. For example, the first calculation unit 22 extracts the feature quantities included in the temporal changes in the pressure data. For example, the first calculation unit 22 extracts the feature quantities included in the load waveforms. For example, the first calculation unit 22 extracts feature quantities including at least one of the following: peak values and a dip value of the load waveforms. The first calculation unit 22 extracts a feature quantity vector as a feature, the feature quantity vector including elements that are the values of a first peak, a second peak, and dip included in the load waveforms, and body weight information.

Using the gait data as sample data, the second calculation unit 24 learns the correlation between the feature quantities extracted by the first calculation unit 22 and the body weight information about the walking person, and generates a learning model.

The estimation unit 26 inputs the gait data of the estimation target to the learning model, and estimates the body weight information associated with the gait data of the estimation target.

An example configuration of the body weight estimation system 20 of the present example embodiment has been described so far. However, the configuration of the body weight estimation system 20 shown in FIG. 12 is merely an example, and the configuration of the body weight estimation system 20 according to the present example embodiment does not need to remain as it is.

The body weight estimation system 20 may include a gait data generation unit that detects pressure data with pressure sensors installed on the soles of the walking person, extracts gait data from the detected pressure data, and stores the gait data into a database. In this case, the data reception unit 21 receives the gait data to be stored into the database.

The body weight estimation system 20 may also include a first storage unit in which the feature quantities extracted by the first calculation unit 22 are stored, and a second storage unit in which the learning model generated by the second calculation unit 24 is stored. In this case, the second calculation unit 24 generates the learning model by using the feature quantities stored in the first storage unit, and stores the generated learning model into the second storage unit. The body weight estimation system 20 may also include a data transmission unit that transmits body weight data including the body weight information estimated by the estimation unit 26.

As described above, a body weight estimation system of the present example embodiment receives gait data including the gait characteristics of a walking person, and extracts the feature quantities based on the gait characteristics of the walking person from the gait data. Using the gait data as sample data, the body weight estimation system of the present example embodiment also learns the correlation between the feature quantities extracted by the first calculation unit and body weight information about the walking person, and generates a learning model. The body weight estimation system of the present example embodiment then inputs the gait data of the estimation target to the learning model, and estimates the body weight information associated with the gait data of the estimation target.

For example, in the learning mode, the body weight estimation system of the present example embodiment receives gait data and the body weight information associated with the gait data, and generates a learning model using the received body weight information and the feature quantities extracted from the gait data. In the measurement mode, the body weight estimation system of the present example embodiment estimates the body weight information associated with the gait data, using the learning model.

The body weight estimation system of the present example embodiment learns the correlation between the feature quantities indicating the characteristics of the gait data and the body weight information. As the gait data acquired from a biological region other than the specific regions is applied to the learning model acquired by learning, it is possible to estimate the body weight, using the gait data acquired from the biological region other than the specific regions. That is, as the body weight estimation system of the present example embodiment uses the learning model, the body weight can be estimated more accurately than with the body weight estimation system of the first example embodiment.

### Example

Next, procedures for causing a body weight estimation device according to each example embodiment of the present invention to record feature quantities indicating the gait characteristics of the subject are described, with reference to drawings. In this example, foot pressure data of the subject during walking was measured with a foot pressure measuring device with a resolution of one kilogram.

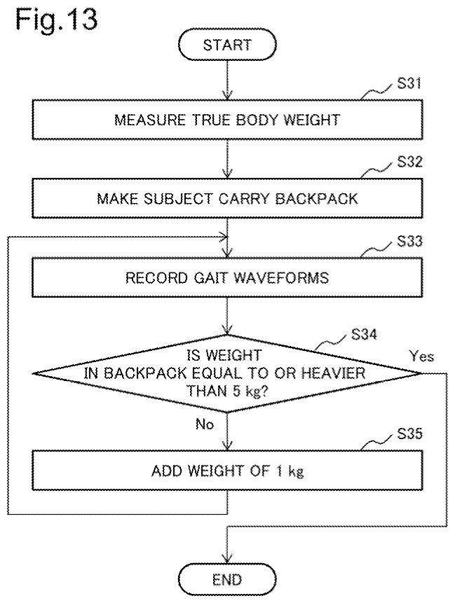

FIG. 13 is a flowchart for explaining the procedures for recording feature quantities in this example. The principal operator of the operation described below was the operator who handled the body weight estimation device of each example embodiment.

In FIG. 13, the operator first measured the body weight of the subject (step S31). At this point, body weight data corresponding to the true body weight of the subject was obtained. The operator stored the body weight data corresponding to the true body weight of the subject into the body weight estimation device.

The operator then made the subject carry a backpack (step S32). In the initial state, the backpack was empty. In this example, weights were added to the backpack being carried by the subject, to increase the body weight of the subject in an artificial manner. In this manner, a variation of response variables was increased. In this example, weights of one kilogram were added to the inside of the backpack one by one, and eventually, the total weight was increased to five kilograms.

The operator then made the subject carrying the backpack walk, and caused the body weight estimation device to record the gait waveforms (step S33). The operator caused the body weight estimation device to record the gait waveforms with respect to each weight by changing the body weight of the subject in the range of zero to five kilograms, and extract feature quantities from the gait waveforms.

If the weight in the backpack was less than five kilograms (No in step S34), a weight was added to the inside of the backpack (step S35). After step S35, the operation returned to step S33, to continue the recording of gait waveforms. If the weight in the backpack was 5 kg or greater (Yes in step S34), on the other hand, the process according to the flowchart in FIG. 13 came to an end.

The procedures for recording feature quantities have been described so far. However, the above process according to the flowchart in FIG. 13 is merely an example, and does not limit procedures for recording feature quantities according to each example embodiment.

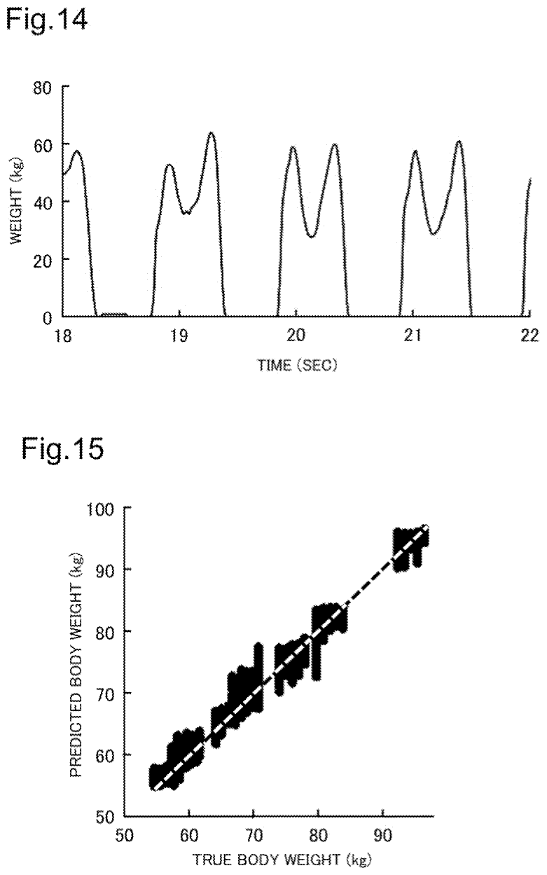

FIG. 14 is an example of the gait waveforms recorded when the subject was actually walking. As shown in FIG. 14, waveforms close to the ideal waveforms were obtained. The body weight estimation device was made to extract feature quantities from the gait waveforms shown in FIG. 14. Thus, the feature quantity vectors of the subject were obtained.

In this example, a feature quantity importance analysis using a random forest learning device was conducted. As a result, it was found that the mean value of the first peak and the second peak, and the time integral value of the gait waveforms of steps were important. Therefore, it is beneficial that the body weight estimation device of each example embodiment is designed to be capable of measuring either the first peak and the second peak, or the integral value of gait waveforms.

FIG. 15 shows the correlation between predicted body weights and the true body weights obtained by recording feature quantities of eight subjects in accordance with the flowchart shown in FIG. 13. In the example shown in FIG. 15, the body weights of the eight subjects were artificially increased, and the gait waveforms were measured at different body weights. The feature quantities were totaled for creating a learning model, and the accuracy of the estimation results was evaluated by a cross-validation method. Specifically, 15% of the teaching data for learning was randomly extracted, and the remaining 85% of the data was used to create learning data. After that, the obtained 15% data was input to the learning device, the output predicted body weight data was compared with the true body weights, and accuracy was evaluated on the basis of the mean square error of the differences from the true body weights. As a result, the mean square error was 1.16 kilograms, which was confirmed to be close to the true resolution.

### Hardware

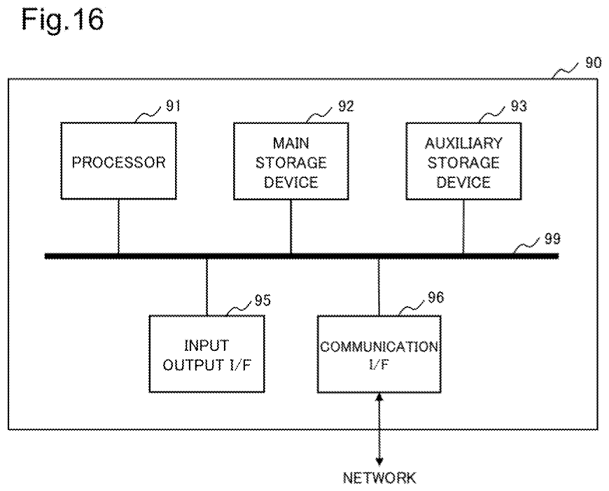

A hardware configuration for forming a body weight estimation system according to each example embodiment of the present invention is now described, with an information processing apparatus 90 in FIG. 16 being an example. However, the information processing apparatus 90 shown in FIG. 16 is merely an example configuration for performing processes in the body weight estimation system of each example embodiment, and does not limit the scope of the present invention.

As shown in FIG. 16, the information processing apparatus 90 includes a processor 91, a main storage device 92, an auxiliary storage device 93, an input/output interface 95, and a communication interface 96. In FIG. 16, “interface” is abbreviated as “I/F”. The processor 91, the main storage device 92, the auxiliary storage device 93, the input/output interface 95, and the communication interface 96 are connected to one another via a bus 99 in such a way as to be capable of data communication. The processor 91, the main storage device 92, the auxiliary storage device 93, and the input/output interface 95 are also connected to a network such as the Internet or an intranet via the communication interface 96.

The processor 91 loads a program stored in the auxiliary storage device 93 or the like into the main storage device 92, and executes the loaded program. In the present example embodiment, a software program installed in the information processing apparatus 90 may be used.

The processor 91 performs the process in the body weight estimation system according to the present example embodiment.

The main storage device 92 has a region in which a program is loaded. The main storage device 92 may be a volatile memory such as a dynamic random access memory (DRAM), for example. A nonvolatile memory such as a magnetoresistive random access memory (MRAM) may be composed and/or added as the main storage device 92.

The auxiliary storage device 93 stores various kinds of data. The auxiliary storage device 93 is formed with a local disk such as a hard disk or a flash memory. However, the main storage device 92 may be designed to store various kinds of data, and the auxiliary storage device 93 may not be provided.

The input/output interface 95 is an interface for connecting the information processing apparatus 90 to peripheral devices. The communication interface 96 is an interface for connecting to an external system or device via a network such as the Internet or an intranet, in accordance with standards or specifications. The input/output interface 95 and the communication interface 96 may be shared as an interface for connecting to an external device.

An input device such as a keyboard, a mouse, or a touch panel may be connected to the information processing apparatus 90 as needed. These input devices are used for inputting information and settings. In a case where a touch panel is used as an input device, the display screen of a display device may also serve as the interface with the input device. Data communication between the processor 91 and the input device may be mediated by the input/output interface 95.

The information processing apparatus 90 may also be equipped with a display device for displaying information. In a case where a display device is provided, the information processing apparatus 90 preferably includes a display controller (not shown) for controlling display on the display device. The display device may be connected to the information processing apparatus 90 via the input/output interface 95.

The information processing apparatus 90 may also be equipped with a disk drive if necessary. The disk drive is connected to the bus 99. Between the processor 91 and a recording medium (a program recording medium) that is not shown in the drawing, the disk drive mediates reading data or a program from the recording medium, writing of results of processing performed by the information processing apparatus 90 into the recording medium, and the like. The recording medium can be formed with an optical recording medium such as a compact disc (CD) or a digital versatile disc (DVD), for example. Alternatively, the recording medium may be formed with a semiconductor recording medium such as a universal serial bus (USB) memory or a secure digital (SD) card, a magnetic recording medium such as a flexible disk, or some other recording medium.

An example hardware configuration for achieving a body weight estimation system according to each example embodiment of the present invention has been described so far. However, the hardware configuration shown in FIG. 16 is merely an example hardware configuration for performing an arithmetic process in the body weight estimation system according to each example embodiment, and does not limit the scope of the present invention. A program for causing a computer to execute a process related to a body weight estimation system according to each example embodiment is also included in the scope of the present invention. Further, a program recording medium storing a program according to each example embodiment is also included in the scope of the present invention.

The components of the body weight estimation system of each example embodiment may be combined as appropriate. The components of the body weight estimation system of each example embodiment may be formed by software, or may be formed by circuitry.

While the present invention has been particularly shown and described with reference to example embodiments thereof, the present invention is not limited to these example embodiments. It will be understood by those of ordinary skill in the art that various changes in form and details may be made therein without departing from the spirit and scope of the present invention as defined by the claims.

### REFERENCE SIGNS LIST

1 body weight estimation system
11 data acquisition device
12 data collection device
13 body weight estimation device
14 transmission device
110 data acquisition unit
111 main frame
112 sensor unit
115 sensor data transmission unit
121 sensor data reception unit
122 gait data extraction unit
123 body weight information input unit
124 database
131 data reception unit
132 first calculation unit
133 feature quantity memory unit
134 second calculation unit
135 learning model memory unit
136 estimation unit
137 data transmission unit

What is claimed is:

1. A body weight estimation system comprising:
a data receiver configured to receive gait data including gait characteristics of a walking person;
at least one memory storing instructions; and
at least one processor connected to the at least one memory and configured to execute the instructions to:
extract a feature quantity based on the gait characteristics of the walking person from the gait data;
generate a learning model by learning a correlation between the extracted feature quantity and body weight information about the walking person, using the gait data as sample data; and
estimate the body weight information associated with the gait data of an estimation target by inputting the gait data of the estimation target to the learning model.

2. The body weight estimation system according to claim 1, wherein
the data receiver is configured to receive the gait data that is data related to a temporal change in pressure data of a sole of the walking person, and
the at least one processor is configured to execute the instructions to extract a feature quantity included in the temporal change in the pressure data.

3. The body weight estimation system according to claim 1, wherein
the data receiver is configured to receive the gait data that is a load waveform based on a temporal change in pressure data of a sole of the walking person, and
the at least one processor is configured to execute the instructions to extract a feature quantity included in the load waveform.

4. The body weight estimation system according to claim 3, wherein
the at least one processor is configured to execute the instructions to extract a feature quantity including at least one of a peak value and a dip value of the load waveform.

5. The body weight estimation system according to claim 4, wherein
the at least one processor is configured to execute the instructions to extract the feature quantity that is a feature quantity vector having elements that are values of a first peak, a second peak, and a dip included in the load waveform, and the body weight information.

6. The body weight estimation system according to claim 2, wherein
the at least one processor is configured to execute the instructions to
detect the pressure data with a pressure sensor installed on the sole of the walking person,
extract the gait data from the detected pressure data, and
store the extracted gait data into a database, and
the data receiver is configured to receive the gait data to be stored into the database.

7. The body weight estimation system according to claim 1, further comprising:
a first storage configured to store the extracted feature quantity;
a second storage configured to store the generated learning model; and
a data transmitter configured to transmit body weight data including the estimated body weight information, wherein
the at least one processor is configured to execute the instructions to
generate the learning model using the feature quantity stored in the first storage, and
store the generated learning model into the second storage.

8. A body weight estimation method comprising:
receiving gait data including gait characteristics of a walking person;
extracting a feature quantity based on the gait characteristics of the walking person from the gait data;
generating a learning model by learning a correlation between the extracted feature quantity and body weight information about the walking person, using the gait data as sample data; and
estimating the body weight information associated with the gait data of an estimation target by inputting the gait data of the estimation target to the learning model.

9. The body weight estimation method according to claim 8, wherein,
in a learning mode,
receiving the gait data and the body weight information associated with the gait data, and
generate the learning model by using the received body weight information and the feature quantity extracted from the gait data, and,
in a measurement mode,
estimating the body weight information associated with the gait data by using the learning model.

10. A non-transient program recording medium storing a program for causing a computer to perform:
a process for receiving gait data including gait characteristics of a walking person;
a process for extracting a feature quantity based on the gait characteristics of the walking person from the gait data;
a process for generating a learning model by learning a correlation between the extracted feature quantity and body weight information about the walking person, using the gait data as sample data; and
a process for estimating the body weight information associated with the gait data of an estimation target by inputting the gait data of the estimation target to the learning model.
